(19) United States
(12) Patent Application Publication
(10) Pub. No.: US 2026/0357599 A1
(43) Pub. Date: Nov. 12, 2026

(54) APPARATUS AND METHOD FOR SOUNDPROOF THERAPY BOOTH

(71) Applicant: William A. Mabrey, Okeechobee, FL (US)

(72) Inventor: William A. Mabrey, Okeechobee, FL (US)

(21) Appl. No.: 19/671,987

(22) Filed: May 8, 2026

Related U.S. Application Data
(60) Provisional application No. 63/802,757, filed on May 9, 2025.

(51) Int. Cl. E04H 1/12; A61B 5/00; A61B 5/11; G06V 40/16; G09B 5/02; G16H 20/00; H04N 23/661; H04N 23/695; H04N 23/90

(52) U.S. Cl. CPC E04H 1/125; A61B 5/1114; A61B 5/4803; A61B 5/7465; G06V 40/166; G06V 40/171; H04N 23/661; H04N 23/695; H04N 23/90; G09B 5/02; G16H 20/00

(57) ABSTRACT

A system and method for performing comprehensive speech therapy within a soundproof enclosure. A user is positioned within an acoustically isolated booth, where a first camera captures full-face video and a pan-tilt-zoom (PTZ) camera captures close-up imagery of oral articulators. Speech audio is recorded using a microphone in a noise-controlled environment. A computing controller simultaneously processes the captured audio and video data to analyze articulation patterns, oral motor function, voice quality, and fluency characteristics. Multi-domain assessment data is generated based on the analysis, and real-time feedback is provided to the user via a display and audio output devices. The system enables detailed oral motor evaluation using video-based analysis without requiring medical imaging equipment. A sound-baffled ventilation system maintains airflow within the enclosure while preserving acoustic isolation, thereby supporting accurate speech capture and continuous operation.

### CROSS-REFERENCE TO RELATED APPLICATION

The present application claims priority to, and the benefit of, U.S. Provisional Application No. 63/802,757 which was filed on May 9, 2025 and is incorporated herein by reference in its entirety.

### FIELD OF THE INVENTION

The impetus of the invention generally relates to therapy systems, however, other service delivery can be supported such as; occupational therapy, psychotherapy, counseling, tutoring, reading specialist, ELA teachers, diagnostic testing and swallowing evaluation. More specifically, the present invention relates to a soundproof speech therapy booth configured to facilitate remote teletherapy (i.e., telehealth link) and multi-domain speech therapy within an acoustically controlled environment. The invention comprises a modular, self-contained enclosure defined by a plurality of multi-layer wall panels forming an interior space designed to minimize ambient noise interference. The system further includes a dual-camera capture arrangement comprising a first camera configured to capture full-face video of a user and a second camera comprising a pan-tilt-zoom (PTZ) camera configured to capture high-resolution close-up imagery of oral articulators. A microphone is disposed within the enclosure to record speech audio under controlled acoustic conditions. A computing controller is operatively connected to the cameras and the microphone and is configured to simultaneously process the captured audio and video data to generate multi-domain speech assessment data and provide real-time feedback to the user. Accordingly, the present disclosure makes specific reference thereto. Nonetheless, it is to be appreciated that aspects of the present invention are also equally applicable to other like applications, devices, and methods of manufacture.

### BACKGROUND

By way of background, there is a widespread shortage of qualified speech-language pathologists and speech-language pathology assistants across multiple service settings, including, hospitals, universities, schools, clinics, State and community early intervention programs. The shortage is driven in part by limited funding resources as well as challenges in attracting and retaining adequately trained professionals. As a result, many individuals who qualify for speech and language services are unable to receive timely and consistent therapy.

The lack of access to speech-language services is particularly critical in educational settings. This is particularly true for students and patients who live in rural areas where it is even harder to attract qualified candidates. Students who require such services but do not receive appropriate intervention may experience significant long-term consequences, including impaired academic performance, and diminished lifelong social, educational and vocational opportunities. It has been reported that a substantial percentage of incarcerated individuals (estimated to be approximately 80%) received or qualified for speech and language therapy. Having not received treatment during earlier stages of development, highlighting's the broader societal impact of unmet needs in this field.

Additionally, there are legal obligations associated with providing speech-language services. For example, in school systems, eligible students are provided with an Individualized Education Program (IEP), which is a legally binding document that mandates the provision of specified services. Failure to deliver these services may result in non-compliance with legal requirements, thereby exposing institutions, such as school districts, to potential legal and regulatory consequences. Accordingly, there exists a need for systems and methods that improve access to speech-language services, which address resource limitations and ensure compliance.

Therefore, there exists a long-felt need in the art for an improved system for delivering speech-language therapy services that overcomes the limitations associated with shortages of qualified SLP and SLPA. There is a long-felt need for a solution that enables individuals, particularly students in educational settings, to receive timely and consistent speech-language services despite resource constraints. Additionally, there exists a need for a system that minimizes the impact of workforce shortages by enabling supported remote access. Moreover, there is a need for a supportive environment that provides controlled acoustic conditions to ensure a reliable audio capture and analysis. Finally, there is a need for a system that supports compliance towards legally mandated service requirements, such as IEPs/treatment plans, while reducing the burden on institutions responsible for providing such services.

The subject matter disclosed and claimed herein, in one embodiment, comprises a soundproof speech booth system configured to facilitate remote speech-language therapy and comprehensive multi-domain for service delivery. The system includes a soundproof enclosure defining an acoustically isolated interior space configured to minimize ambient noise interference. The system further includes a first camera configured to capture a full-face video stream of a user and a second camera comprising a pan-tilt-zoom (PTZ) camera configured to capture close-up imagery of oral articulators. A microphone is disposed within the enclosure to record speech audio under controlled acoustic conditions. A computing controller is operatively connected to the cameras and the microphone and is configured to simultaneously process the captured data to generate multi-domain speech assessment data and provide real-time feedback to the user.

In one embodiment, the speech therapy booth system further includes a ventilation system configured to provide continuous airflow within the enclosure while preserving acoustic isolation. The ventilation system includes an air intake vent and an exhaust vent, each incorporating a sound-baffled assembly defining an airflow pathway lined with acoustic material. The system can further include a display for presenting feedback to the user, speakers or a headset for audio output, and a network interface configured to enable communication with a remote speech-language pathologist or cloud-based processing system. The computing controller is configured to perform simultaneous analysis of speech and video data across multiple speech domains, including articulation, phonology, fluency, oral motor function, voice, receptive language, expressive language, pragmatics, and hearing.

In this manner, the speech assessment booth system of the present invention overcomes long-standing deficiencies in the field by providing a scalable, accessible, and technology-enabled solution for speech-language therapy. The invention enables high-quality speech analysis through the use of dual-camera capture and acoustically isolated audio recording, while reducing reliance on in-person clinical resources. The system supports remote therapy delivery, improves access to services for underserved populations, and facilitates compliance with mandated service requirements. As a result, the invention provides improved clinical effectiveness, operational efficiency, and long-term accessibility for speech-language services.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a method of performing comprehensive speech language services. The method includes positioning a user within a soundproof enclosure configured to provide an acoustically isolated environment. The method further includes capturing a first video stream of a face of the user using a first camera and capturing a second video stream comprising a close-up view of oral articulators of the user using a pan-tilt-zoom (PTZ) camera. The method also includes recording speech audio of the user within the acoustically isolated environment. The captured first video stream, second video stream, and speech audio are simultaneously analyzed to determine articulation patterns, oral motor function, voice quality, and fluency characteristics. Based on the simultaneous analysis, multi-domain assessment data is generated, and real-time feedback is provided to the user.

In another embodiment, a speech therapy system is disclosed. The system includes a soundproof enclosure defined by a plurality of wall panels forming an acoustically isolated interior space. The system further includes a first camera configured to capture a full-face video stream of a user and a pan-tilt-zoom (PTZ) camera configured to capture close-up imagery of oral articulators of the user. A microphone is provided for recording speech audio within the acoustically isolated interior space. The system also includes a computing controller configured to receive the captured video streams and the speech audio and to simultaneously analyze the received data to generate multi-domain speech assessment data. The system further includes a display configured to present real-time feedback to the user based on the generated assessment data.

In a further embodiment, a speech assessment apparatus is disclosed. The apparatus includes a first image capture device positioned to obtain a frontal view of a user and a second image capture device comprising a pan-tilt-zoom camera positioned to obtain an angled close-up view of oral articulators of the user. The apparatus further includes a processing system configured to receive data from the first image capture device and the second image capture device. The processing system is further configured to process the received image data together with recorded speech audio. The processing system performs simultaneous analysis of the data to enable speech assessment based on both facial and oral articulator information.

In still another embodiment, a soundproof speech booth is disclosed. The booth includes a plurality of wall panels forming an enclosed interior space having acoustic isolation properties. The booth further includes an air intake vent disposed at a lower portion of the enclosure and an exhaust vent disposed at an upper portion of the enclosure. Each of the air intake vent and the exhaust vent includes a sound-baffled assembly comprising a plurality of directional baffles defining a labyrinth airflow pathway. The interior surfaces of the airflow pathway are lined with acoustic material. The ventilation system is configured to provide airflow through the enclosure while maintaining acoustic isolation for accurate speech recording.

In some embodiments, a speech assessment system is disclosed. The system includes a microphone configured to capture speech audio, a first camera configured to capture facial video, and a second camera configured to capture close-up oral articulator video. The system further includes a computing controller configured to receive the captured data from the microphone and the cameras. The computing controller is configured to simultaneously analyze the captured data to generate assessment data across multiple speech domains. The multiple speech domains include articulation, phonology, fluency, oral motor function, voice, receptive language, expressive language, pragmatics, and hearing.

In one aspect, each of the wall panels comprises a multi-layer structure. The multi-layer structure includes an acoustic foam layer disposed on an interior surface, an insulation layer within a wall cavity, a structural wood frame, and an exterior panel. The multi-layer structure is configured to provide both sound absorption and sound blocking (i.e., soundproofing) within the enclosure.

The pan-tilt-zoom camera includes at least 20× optical zoom capability. The PTZ camera is configured to capture high-resolution imagery of lips, teeth, tongue, and jaw of the user. The captured imagery is used for detailed articulation pattern analysis and oral motor assessment.

The oral motor function is determined from the close-up imagery captured by the PTZ camera. The determination is performed without the use of medical imaging systems. The medical imaging systems include computed tomography and video fluoroscopy.

The air intake vent and the exhaust vent are positioned diagonally opposite each other within the enclosure. This positioning creates a cross-ventilation airflow path across the interior space. The cross-ventilation ensures circulation of air throughout the enclosure.

A relay module is connected to the computing controller and is configured to control operation of one or more electrical components. The electrical components include at least one of an exhaust fan, an intake fan, and a lighting system.

The computing controller is configured to provide real-time feedback to the user. The real-time feedback includes visual feedback provided via a display and audio feedback provided via speakers or a headset. The feedback is generated based on the multi-domain assessment data.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective cutaway view of a speech therapy booth of the present invention in accordance with the disclosed structure;

FIG. 2 illustrates a top-plan view of the speech therapy booth of FIG. 1, showing the interior floor plan layout, in accordance with the disclosed structure;

FIG. 3 illustrates a front elevation view of the speech therapy booth showing the exterior front face of the booth in accordance with the disclosed structure;

FIG. 4 illustrates a side elevation cross-sectional view of the speech therapy booth of the present invention in accordance with the disclosed structure;

FIG. 5 illustrates a schematic wiring diagram of the electronic control system of the speech therapy booth of the present invention in accordance with the disclosed structure;

FIG. 6 illustrates an enlarged cross-sectional detail view of the automatic door sweep assembly of the door of the speech therapy booth, in accordance with the disclosed structure;

FIG. 7 illustrates an enlarged cross-sectional detail view of an alternative bottom door seal embodiment of the door of the speech therapy booth in accordance with the disclosed structure;

FIG. 8 illustrates an enlarged horizontal cross-sectional detail view of the door frame seal assembly of the speech therapy booth in accordance with the disclosed structure; and

FIG. 9 illustrates a flowchart depicting a method of performing multi-domain speech assessment using the speech therapy booth in accordance with an embodiment of the present invention.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an improved system for delivering speech-language therapy services that overcomes the limitations associated with shortages of qualified SLP and SPLA. There is a long-felt need for a solution that enables individuals, particularly students in educational settings, to receive timely and consistent speech-language services despite resource constraints. Additionally, there exists a need for a system that minimizes the impact of workforce shortages by enabling remote or automated therapy while maintaining clinical accuracy. Moreover, there is a need for a speech therapy environment that provides controlled acoustic conditions to ensure reliable audio capture and analysis. Finally, there is a need for a system that supports compliance with legally mandated service requirements, including Individualized Education Programs (IEPs), while reducing the burden on institutions providing such services.

The present invention, in one exemplary embodiment, is a speech assessment system. The system includes a microphone configured to capture speech audio, a first camera configured to capture facial video, and a second camera configured to capture close-up oral articulator video. The system further includes a computing controller configured to receive the captured data from the microphone and the cameras. The computing controller is configured to simultaneously analyze the captured data to generate assessment data across multiple speech domains. The multiple speech domains include articulation, fluency, oral motor function, voice, receptive language, expressive language, pragmatics, and hearing.

Reference will now be made in detail to the present preferred embodiments of the invention, examples of which are illustrated in the accompanying drawings. Wherever possible, the same reference numerals are used in the drawings and the description to refer to the same or like parts.

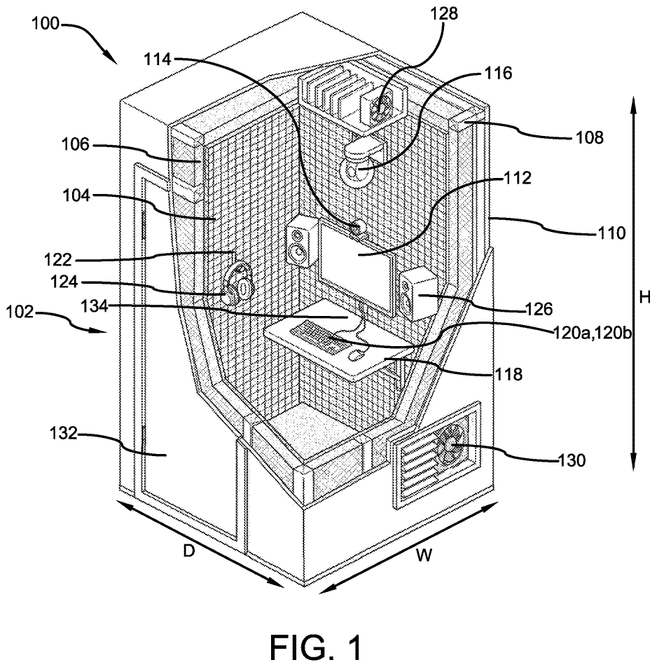

Referring initially to the drawings, FIG. 1 illustrates a perspective cutaway view of a speech therapy booth of the present invention in accordance with the disclosed structure. The speech therapy booth 100 is configured as a modular, self-contained, soundproof enclosure designed to facilitate remote speech-language therapy sessions via teletherapy or telehealth and AI-assisted speech assessment, accommodating up to two users simultaneously within the interior space of the speech therapy booth 100.

More specifically, the speech therapy booth 100 has an exterior width (“W”) of approximately four to six feet, an exterior depth (“D”) of approximately four to five feet, and an exterior height (“H”) of approximately seven to nine feet, thereby providing an interior floor space of approximately sixteen (16) to twenty (20) square feet. The overall enclosure structure is defined by a plurality of wall panels 102 forming the exterior shell of the speech therapy booth 100, wherein each wall panel 102 is constructed as a modular, multi-layer composite assembly having a total wall thickness of approximately two (2) to four (4) inches. The plurality of wall panels 102 comprise a back wall panel, a left wall panel, a right wall panel, and a front wall panel.

The modular construction of the wall panels 102 enables panel-to-panel connection via cam locks or bolts, with corner joints reinforced by metal L-brackets, and all inter-panel joints and seams sealed with acoustic caulk to prevent sound transmission (i.e., sound-proof) through gaps between panels. The speech therapy booth 100 is anchored to the installation surface via anchor bolts disposed at the base of the wall panels 102, with vibration isolation pads interposed between the base of the speech therapy booth 100 and the installation surface to attenuate structure-borne vibration transmission.

Each wall panel 102 is constructed as a composite, multi-layer assembly comprising an acoustic foam panel 104 disposed on the innermost surface of each wall panel 102, wherein the acoustic foam panel 104 comprises approximately one inch (1″) of pyramid-style or wedge-style acoustic foam providing sound absorption across the interior space of the speech therapy booth 100. The foam insulation 106 comprises approximately one inch (1″) of R-13 fiberglass or mineral wool batt insulation providing combined thermal and acoustic insulation within the wall cavity.

A frame 108 can comprise two-by-two (2×2) dimensional lumber having actual dimensions of approximately one and one-half inches by one and one-half inches (1.5″×1.5″) form the structural skeleton of each wall panel 102. In some embodiments, the wall panel 102 can include a mass loaded vinyl (MLV) having a surface density of approximately one pound per square foot (1 lb./sq ft) disposed between the acoustic foam panel 104 and the foam insulation 106 for enhanced sound blocking (i.e., soundproofing).

An exterior panel 110 is disposed on the outermost surface of each wall panel 102 and can comprise three-quarter inch (¾″) plywood or medium-density fiberboard (MDF) panel material forming the finished exterior surface of the speech therapy booth 100, wherein the exterior panel 110 is attached to the frame 108 using one-and-one-quarter inch (1¼″) screws, and wherein the wall panels 102 are assembled to the frame 108 using two-and-one-half inch (2½″) screws with corner joints further reinforced by sixteen (16) metal L-brackets.

A monitor 112 is mounted within the interior space of the speech therapy booth 100 and the monitor 112 can comprise an anti-glare 1080p display, positioned at approximately forty-eight inches (48″) above the floor of the speech therapy booth 100 corresponding to the seated eye level of a user. Preferably, the monitor 112 is mounted on the interior back wall surface of the speech therapy booth 100 via a monitor wall mount, configured to display teletherapy or telehealth link) session content, real-time AI-generated speech assessment feedback, therapy exercises, and video conferencing content to a user seated within the speech therapy booth 100.

A webcam 114 is mounted proximate to and above the monitor 112, comprising a standard 1080p USB webcam configured to capture a full-face video stream of the user at a frame rate sufficient for real-time facial expression analysis, eye contact tracking, and video conferencing during a therapy session, wherein the webcam 114 constitutes a first image capture device of the dual-camera assessment system of the speech therapy booth 100.

A pan-tilt-zoom (PTZ) camera 116 is mounted on the interior wall of the speech therapy booth 100 at a camera mounting height positioned to provide an angled view toward the user's seated position. The PTZ camera 116 can have at least twenty times (20×) optical zoom capability and pan-tilt control via USB or network connection, constituting a second image capture device of the dual-camera assessment system of the speech therapy booth 100, wherein the PTZ camera 116 is configured to capture high-resolution close-up video imagery of the user's oral articulators including the lips, teeth, tongue, and jaw, enabling articulation pattern analysis, oral motor function assessment, lip position tracking, jaw movement analysis, and tongue visibility detection during a speech therapy session, thereby enabling oral motor assessment without the requirement of clinical imaging equipment such as computed tomography (CT) scanners or video fluoroscopy systems.

A desk 118 is mounted at a standard seated working height of approximately thirty inches (30″) above the floor of the speech therapy booth 100, providing a work surface of approximately thirty-six inches (36″) in width by eighteen inches (18″) in depth for the user. A keyboard and mouse assembly 120a, 120b is disposed on the surface of the desk 118, comprising a USB wired keyboard and USB wired mouse enabling user interaction with the computing system of the speech therapy booth 100.

A headphone hook 122 is mounted and is configured to store and retain a headphone 124 when not in use. The headphone 124 is suspended from the headphone hook 122 and comprises a USB headset configured to provide private audio output to the user and to serve as a backup audio input device during a therapy session.

A speaker 126 is mounted and can comprise a USB-powered speaker or soundbar configured to deliver audio output from a remote speech-language pathologist or from the AI-assisted therapy processing system to the user within the speech therapy booth 100.

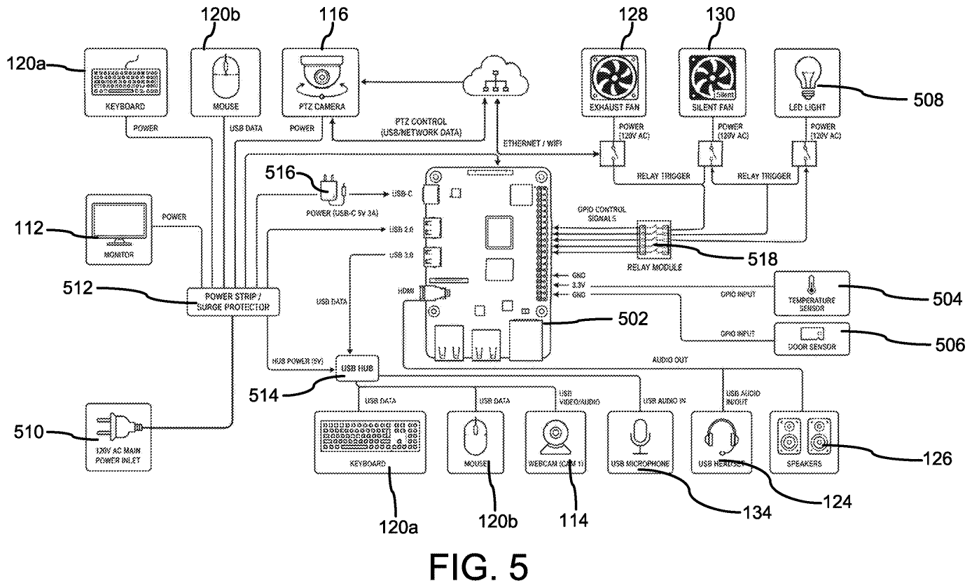

A USB condenser microphone 134 is further disposed on the surface of the desk 118 as the primary audio capture device for recording the speech of the user within the acoustically isolated interior space of the speech therapy booth 100. An LED light panel (shown in FIG. 5) is mounted to the ceiling of the speech therapy booth 100, comprising a dimmable warm-white LED panel providing interior illumination of the speech therapy booth 100, controlled via a relay module connected to the central computing controller of the speech therapy booth 100.

The speech therapy booth 100 further comprises a ventilation system configured to provide continuous airflow through the interior space of the speech therapy booth 100 while preserving the acoustic isolation properties thereof. An exhaust fan 128 is mounted at or near the ceiling-wall junction at the upper right corner of the speech therapy booth 100, comprising a fan unit rated at between fifty and seventy-five cubic feet per minute (50-75 CFM) airflow capacity for single-occupant use. The exhaust fan 128 is configured to expel heated air from the interior space of the speech therapy booth 100 to the exterior environment through a sound-baffled exhaust assembly comprising a baffle box having overall dimensions of approximately twelve inches by twelve inches by eight inches deep (12″×12″×8″) constructed from three-quarter inch (¾″) medium-density fiberboard (MDF) or plywood, lined on all interior surfaces with, for example, one inch (1″) thick acoustic foam, and incorporating a minimum of three to four (3-4) directional baffles defining a labyrinth or maze airflow pathway, wherein the minimum air gap between adjacent baffles is three inches (3″), the exhaust fan 128 being controlled via the relay module of the central computing controller.

An air intake vent 130 incorporating a silent fan is disposed at a lower portion of the left wall panel of the wall panels 102 near the floor level of the speech therapy booth 100, positioned diagonally opposite to the exhaust fan 128 to promote diagonal cross-ventilation airflow traversing the full interior space of the speech therapy booth 100. The air intake vent 130 incorporates a sound baffle assembly of equivalent construction to the exhaust baffle assembly, comprising a baffle maze box constructed from three-quarter inch (¾″) MDF or plywood lined on all interior surfaces with one inch (1″) thick acoustic foam and incorporating a minimum of three to four (3-4) directional baffles with a minimum three inch (3″) air gap between adjacent baffles, with louvered vent grilles disposed on the exterior face and an interior grille disposed on the interior. The ventilation system is designed to provide a minimum of four to six (4-6) air changes per hour within the interior volume of the speech therapy booth 100.

A door 132 is hingedly mounted within a door frame formed in the front wall panel of the wall panels 102 of the speech therapy booth 100. The door 132 comprises a solid-core door panel having minimum thickness of one and three-quarter inches (1¾″), a door width of twenty-eight inches (28″), and a door height of eighty inches (80″), optionally incorporating a layer of mass loaded vinyl (MLV) bonded to the interior face of the door panel for enhanced sound isolation. The door 132 is mounted to the door frame via three (3) heavy-duty ball-bearing hinges providing adjustment capability for precise door alignment and compression seal engagement.

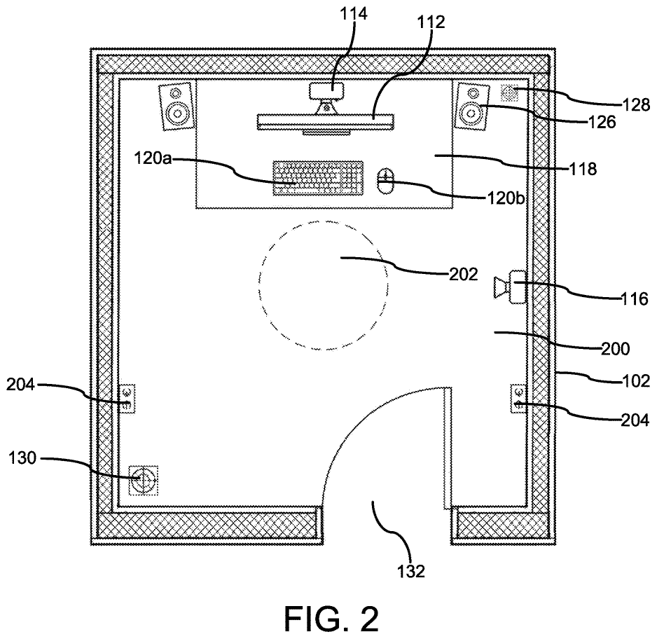

FIG. 2 illustrates a top-plan view of the speech therapy booth 100 of FIG. 1, showing the interior floor plan layout, in accordance with the disclosed structure. As shown in FIG. 2, the interior space 200 of the speech therapy booth 100 is bounded on all four sides by the wall panels 102, wherein the cross-hatched perimeter shown in FIG. 2 represents the two-inch (2″) composite wall construction 102 described with respect to FIG. 1.

The desk 118 is positioned against the back wall of the speech therapy booth 100, centered along the back wall width, and oriented such that the user faces the back wall from a seated position. The desk 118 presents a working surface of approximately thirty-six inches (36″) in width by eighteen inches (18″) in depth as viewed in plan. The keyboard and mouse assembly 120a, 120b is disposed on the surface of the desk 118 in a forward position accessible to the seated user, with the keyboard positioned centrally and the mouse positioned to the right of the keyboard.

The monitor 112 is positioned at the rear of the desk 118 surface against the back wall, centered on the desk 118 as viewed in plan. The webcam 114 is mounted atop the monitor 112, centered on the upper edge thereof, such that the webcam 114 captures a front-facing full-face video stream of the user seated at the desk 118.

The speaker 126 is disposed on each lateral side of the monitor 112 on the desk 118 surface, providing stereo audio output to the user, wherein the two speakers 126 are positioned symmetrically flanking the monitor 112 as viewed in plan. The exhaust fan 128 is disposed at the upper right corner of the interior space of the speech therapy booth 100, mounted at the junction of the right wall panel and the back wall panel of the wall panels 102, as viewed in plan, consistent with its ceiling-wall junction mounting position described with respect to FIG. 1.

In the present embodiment, the PTZ camera 116 is mounted on the right wall panel of the wall panels 102 at a mid-wall position, angled inward toward the seated user position as represented by the dashed circle 202 indicating the chair position of the user within the interior space of the speech therapy booth 100. The PTZ camera 116 is positioned on the right wall panel such that its pan-tilt-zoom field of view is directed toward the user's face and oral articulators when the user is seated at the desk 118 in the chair position 202.

The chair position 202 is shown as a dashed circle centered within the interior floor space of the speech therapy booth 100 at a distance from the desk 118 consistent with a standard seated working position, representing the expected position of the user during a therapy session.

The air intake vent 130 incorporating the silent fan is disposed at the lower left corner of the interior space of the speech therapy booth 100, mounted in the left wall panel of the wall panels 102 at floor level, positioned diagonally opposite to the exhaust fan 128 at the upper right corner, establishing a diagonal cross-ventilation airflow path traversing the full interior floor space of the speech therapy booth 100 from the lower left to the upper right as viewed in plan, thereby ensuring airflow circulation across the entire occupied interior space.

The door 132 is shown in plan view at the bottom center of the speech therapy booth 100, formed within the front wall panel of the wall panels 102, having a clear opening width of twenty-eight inches (28″).

Wall outlet receptacles 204 are disposed on the left wall panel and right wall panel of the wall panels 102 at approximately mid-wall positions along the base of the interior walls, providing AC power access to the interior components of the speech therapy booth 100 consistent with the electrical layout described with respect to FIG. 1.

The spatial relationship between the webcam 114, the PTZ camera 116, and the chair position 202, as shown in FIG. 2, illustrates the dual-camera coverage geometry of the speech therapy booth 100, wherein the webcam 114 provides a frontal full-face capture axis directed perpendicularly from the back wall toward the chair position 202, and the PTZ camera 116 provides a lateral angled capture axis directed from the right wall panel toward the oral articulators of the user at the chair position 202, the two capture axes together enabling simultaneous full-face and high-resolution oral articulator video capture during a speech therapy session.

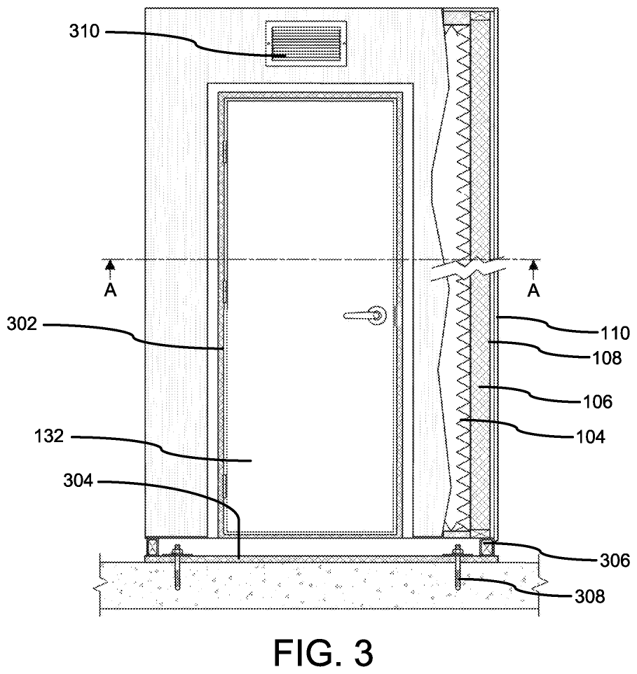

FIG. 3 illustrates a front elevation view of the speech therapy booth showing the exterior front face of the booth in accordance with the disclosed structure. As shown in the sectional portion of FIG. 3, the exterior panel 110 forms the outermost finished surface of the speech therapy booth 100, the frame 108 provides structural support to the wall panel assembly, the foam insulation 106 is disposed within the wall cavity between the frame 108 members providing combined thermal and acoustic insulation of approximately one inch (1″) thickness; and the acoustic foam panel 104 is disposed on the innermost surface of the wall panel providing sound absorption within the interior space of the speech therapy booth 100 of approximately one inch (1″) thickness; all as previously described with respect to FIG. 1.

The door 132 comprises a solid-core door panel as previously described with respect to FIG. 1, shown with a lever handle with cam-lock or deadbolt mechanism disposed on the right side of the door 132 at a standard handle height. A soundproof seal gasket 302 is disposed continuously about the full perimeter of the door frame surrounding the door 132, comprising a combined magnetic seal strip and neoprene compression gasket assembly extending along the top jamb, both side jambs, and interfacing with the bottom door sweep or threshold seal of the door 132, wherein the soundproof seal gasket 302 is configured to create a fully compressed, acoustically sealed closure about the entire perimeter of the door 132 when the door 132 is in the closed position.

The lower portion of FIG. 3 illustrates the foundation connection detail of the speech therapy booth 100 at the base of the front wall panel. A vibration isolation pad 304 is interposed between the base of the wall panels 102 of the speech therapy booth 100 and the foundation 306, extending continuously beneath the full width of the front wall panel of the speech therapy booth 100 as shown in FIG. 3. The vibration isolation pad 304 is configured to attenuate structure-borne vibration transmission between the installation surface and the structural frame of the speech therapy booth 100, thereby preventing floor-transmitted vibrations from compromising the acoustic isolation of the interior space of the speech therapy booth 100 and from interfering with the audio capture and acoustic analysis functions of the speech therapy system. The foundation 306 comprises the installation floor surface upon which the speech therapy booth 100 is positioned, shown in cross-section at the base of FIG. 3. A plurality of anchor bolts 308 extend downward through the base of the wall panels 102, through the vibration isolation pad 304, and into the foundation 306, wherein the anchor bolts 308 are configured to provide permanent or semi-permanent mechanical anchoring of the speech therapy booth 100 to the installation surface, preventing lateral displacement of the speech therapy booth 100 during use.

An exhaust vent grille 310 incorporating adjustable louvers is disposed on the exterior face of the front wall panel of the wall panels 102 at a position near the upper portion of the front wall panel, above the door 132, providing the exterior-facing opening of the exhaust ventilation pathway of the speech therapy booth 100. The exhaust vent grille 310 with louvers is configured to permit the discharge of exhaust airflow from the interior of the speech therapy booth 100 to the exterior environment while providing weather protection against the ingress of precipitation, debris, and exterior contaminants into the exhaust ventilation pathway.

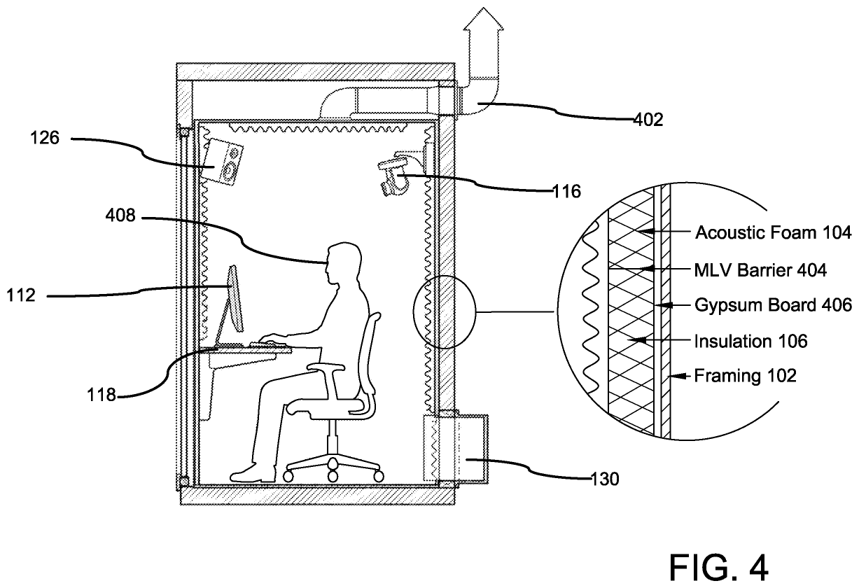

FIG. 4 illustrates a side elevation cross-sectional view of the speech therapy booth of the present invention in accordance with the disclosed structure. A user 408 can be positioned at the desk 118, wherein the desk 118 is mounted at a height of approximately thirty inches (30″) above the interior floor surface. The seated eye level of the user is at approximately forty-eight inches (48″) above the interior floor surface, corresponding to the mounting height of the monitor 112 on the back wall of the speech therapy booth 100. The speaker 126 and the PTZ camera 116 are shown mounted on the interior side wall of the speech therapy booth 100, wherein the PTZ camera 116 is positioned at the camera mounting height angled downward and inward toward the user's oral articulators at the seated position, consistent with the dual-camera geometry described with respect to FIGS. 1 and 2.

An exhaust ventilation routing path 402 exits through the ceiling of the speech therapy booth 100 and discharge upward to the exterior environment. The air intake vent 130 incorporating the silent fan is shown at floor level on the lower portion of the side wall of the speech therapy booth 100, positioned diagonally opposite to the exhaust discharge path, consistent with the diagonal cross-ventilation layout described with respect to FIGS. 1 and 2.

FIG. 4 includes an enlarged circular detail view of the composite wall panel cross-section, illustrating the sequential layers of the wall construction. The acoustic foam panel 104 forms the innermost surface and a mass loaded vinyl (MLV) barrier layer 404 is disposed outward of the acoustic foam panel 104, comprising a sheet of mass loaded vinyl having a surface density of approximately one pound per square foot (1 lb./sq ft) providing supplemental sound blocking (i.e., soundproofing) within the wall assembly. A gypsum board layer 406 is disposed outward of the MLV barrier layer 404 and comprising one-half inch (½″) gypsum board or drywall providing mass and fire resistance to the wall panel assembly. The foam insulation layer 106 is disposed outward of the gypsum board layer 406 within the wall cavity and the framing 102 comprises, for example, the 2×2 lumber wood frame providing structural support to the wall panel assembly, all as previously described with respect to FIG. 1.

FIG. 5 illustrates a schematic wiring diagram of the electronic control system of the speech therapy booth of the present invention in accordance with the disclosed structure. The central computing controller 502 can comprise a Raspberry Pi 4 single-board computer or any other processor serving as the primary control, processing, and communications hub of the speech therapy booth 100. The controller 502 may be equipped with a USB-C power input port, one or more USB 2.0 ports, a plurality of USB 3.0 ports, at least one HDMI output port, a GPIO header for providing a plurality of general-purpose input and output pins operating at 3.3 volt logic levels with ground references, and an integrated Ethernet and Wi-Fi network interface. The controller 502 is configured to receive operating power via a USB-C power input 516 supplied at five volts, three amperes (5V/3A) from a dedicated USB-C power supply connected to the power distribution system of the speech therapy booth 100. The controller 502 is configured to execute the speech therapy session management software, real-time AI-assisted speech assessment processing, peripheral device control, relay switching logic, sensor monitoring, and teletherapy (i.e., telehealth link) video conferencing functions of the speech therapy booth 100, and communicates with external networks, remote speech-language pathologist workstations, and cloud-based AI processing services via its integrated Ethernet and Wi-Fi interface.

Primary electrical power is supplied to the speech therapy booth 100 via a one hundred twenty volt alternating current (120V AC) main power inlet 510, which distributes AC power to a power strip and surge protector 512 mounted under the desk 118. The power strip and surge protector 512 serves as the central AC power distribution point of the speech therapy booth 100, supplying one hundred twenty volt alternating current (120V AC) power independently to the monitor 112, to the USB-C power supply 516 of the controller 502, to the PTZ camera 116 via its dedicated AC power adapter, and to the relay module 518 for downstream relay-switched load distribution, while also supplying five volt direct current (5V DC) hub power to the USB hub 514 via a dedicated hub power connection.

The USB hub 514 receives five volt (5V) bus power from the power strip and surge protector 512 and provides a plurality of USB data and power ports through which the controller 502 communicates with and powers the downstream peripheral devices of the speech therapy booth 100 via the USB 2.0 and USB 3.0 ports of the controller 502.

Peripheral devices connected to the USB hub 514 include the keyboard 120a via a USB data connection, the mouse 120b via a USB data connection, the webcam 114 designated as CAM 1 via a USB video and audio connection transmitting the full-face video stream of the user to the controller 502, the USB microphone 134 via a USB audio input connection serving as the primary speech capture device of the speech therapy booth 100, and the USB headset 124 via a USB audio input and output connection providing private audio output to the user and backup microphone capability.

The speakers 126 receive audio output directly from the audio output port of the controller 502 independently of the USB hub 514. The monitor 112 receives the video signal from the controller 502 via a dedicated HDMI connection to the HDMI output port of the controller 502 and receives AC power independently from the power strip and surge protector 512. The keyboard 120a and mouse 120b receive power from the power strip and surge protector 512 and are also connected through the USB hub 514 to the controller 502, reflecting their combined power and data connectivity within the system.

The PTZ camera 116 receives AC power from the power strip and surge protector 512 via a dedicated power adapter and communicates PTZ positioning control signals and video data to the controller 502 via a USB or network data connection, with network-based PTZ control facilitated through the Ethernet and Wi-Fi interface of the controller 502, enabling remote pan, tilt, and zoom positioning of the PTZ camera 116 by the controller 502 or by a remote speech-language pathologist accessing the speech therapy booth 100 over the network during a therapy session.

The relay module 518 comprises a multi-channel electromechanical or solid-state relay board electrically connected to the GPIO header of the controller 502, receiving relay trigger signals at 3.3 volt logic levels referenced to ground (GND) from dedicated GPIO output pins of the controller 502, and switching one hundred twenty volt alternating current (120V AC) power to relay-controlled loads in response to the trigger signals.

The exhaust fan 128 and the silent fan of the air intake vent 130 each receive 120V AC power through independently switched relay channels of the relay module 518, enabling the controller 502 to independently control the on/off state and, where supported, the speed of each fan in response to temperature sensor readings, door state, session activity, and occupant comfort requirements.

The LED light panel 508 similarly receives 120V AC power through a dedicated relay channel of the relay module 518, enabling controller-triggered on/off switching and dimming of the interior lighting of the speech therapy booth 100 in coordination with session state and door sensor inputs.

A temperature sensor 504, comprising a DHT22 or equivalent digital temperature and humidity sensor, is connected to a dedicated GPIO input pin of the controller 502, continuously providing interior temperature and humidity data to the controller 502 for automated fan speed modulation and occupant comfort management during therapy sessions. A door sensor 506 is connected to a separate GPIO input pin of the controller 502 for providing a binary open or closed state signal to the controller 502 upon each change of state of the door 132. The controller 502 is configured to execute automated responses to door state changes including session pause upon door opening, fan and lighting state adjustments, and session resume upon door closing, thereby integrating the physical access state of the speech therapy booth 100 with the electronic session management functions of the speech therapy system.

In one embodiment, the speech therapy booth system further comprises a method of use for facilitating remote therapy services. In operation, a user enters the interior space of the booth, which is configured for controlled access, and inputs authentication information comprising a user-specific passcode associated with a patient or student profile. Upon validation of the authentication information, the system initiates a teletherapy or telehealth session via a network connection to a remote speech-language pathologist or therapy platform. During the session, the user engages in therapy activities while audio and video data are captured and processed by the system. In certain embodiments, the booth is configured to accommodate up to two users simultaneously within the interior space, thereby enabling concurrent participation in therapy services.

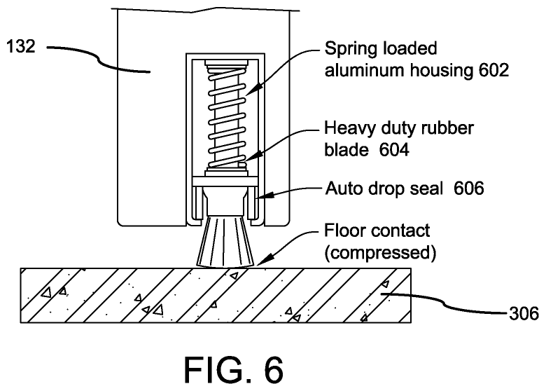

FIG. 6 illustrates an enlarged cross-sectional detail view of the automatic door sweep assembly of the door of the speech therapy booth, in accordance with the disclosed structure. The automatic door sweep assembly is mounted within a routed channel in the bottom edge of the door 132 and comprises a spring-loaded aluminum housing 602, a heavy-duty rubber blade 604, and an auto drop seal 606. The spring-loaded aluminum housing 602 is disposed within the bottom edge of the door 132 and contains a compression spring mechanism configured to bias the auto drop seal 606 and the heavy-duty rubber blade 604 downward toward the foundation 306 upon closure of the door 132.

The heavy-duty rubber blade 604 is carried within the auto drop seal 606 and extends downward from the spring-loaded aluminum housing 602. The heavy-duty rubber blade 604 comprises a flexible, high-durometer rubber material configured to conform to minor surface irregularities of the foundation 306 upon compression, thereby establishing a continuous acoustically sealed contact between the bottom edge of the door 132 and the foundation 306 when the door 132 is in the closed position.

The auto drop seal 606 is mechanically coupled to the spring-loaded aluminum housing 602 such that upon downward movement of the door 132 during closure, the compression spring within the spring-loaded aluminum housing 602 drives the auto drop seal 606 and the heavy-duty rubber blade 604 downward into compressed floor contact against the foundation 306, as indicated by the downward directional arrow shown in FIG. 6.

The horizontal directional arrow shown at floor level in FIG. 6 indicates the direction of door travel during closure, illustrating the operative sequence wherein lateral door movement into the closed position actuates the automatic downward deployment of the auto drop seal 606 and heavy-duty rubber blade 604 into sealing engagement with the foundation 306. The automatic door sweep assembly of FIG. 6 operates in conjunction with the soundproof seal gasket 302 disposed about the top and side perimeters of the door frame as described with respect to FIG. 3, together providing a sealed acoustic enclosure about the complete perimeter of the door 132 when in the closed position.

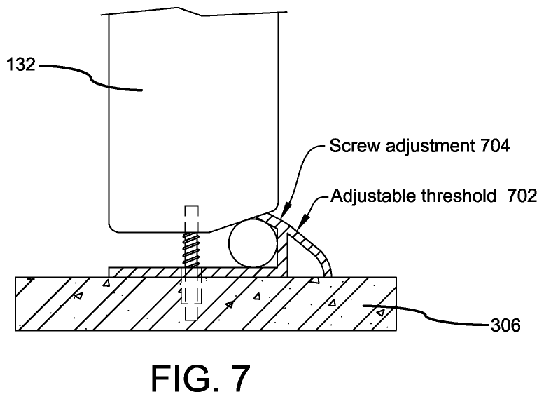

FIG. 7 illustrates an enlarged cross-sectional detail view of an alternative bottom door seal embodiment of the door of the speech therapy booth in accordance with the disclosed structure. As shown in FIG. 7, the adjustable threshold seal assembly comprises an adjustable threshold 702 and a screw adjustment mechanism 704 disposed at the base of the door 132 interface with the foundation 306. The adjustable threshold 702 is mounted to the foundation 306 at the door sill position beneath the closed door 132, having a total height of approximately one-half inch (½″) above the surface of the foundation 306 as indicated in FIG. 7, and incorporates a compressible gasket or roller seal element configured to establish continuous contact with the bottom edge of the door 132 when the door 132 is in the closed position.

The screw adjustment mechanism 704 comprises one or more adjustment screws disposed through the adjustable threshold 702 into the foundation 306, enabling incremental vertical height adjustment of the adjustable threshold 702 relative to the foundation 306 surface, such that the compressible seal element of the adjustable threshold 702 may be precisely calibrated to achieve full compression contact against the bottom edge of the door 132 in the closed position, accommodating variations in floor levelness, door alignment, and seal wear over time.

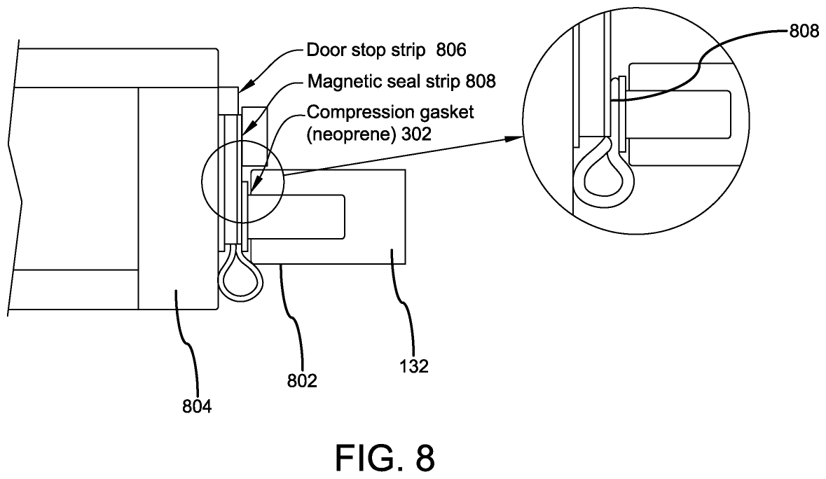

FIG. 8 illustrates an enlarged horizontal cross-sectional detail view of the door frame seal assembly of the speech therapy booth in accordance with the disclosed structure. The door jamb 804 comprises, for example, two-by-six (2×6) dimensional lumber forming the structural door frame of the speech therapy booth 100 within the front wall panel of the wall panels 102. A door stop strip 806 is mounted to the interior face of the door jamb 804, comprising a rigid stop member against which the door edge 802 of the door 132 bears when the door 132 is in the fully closed position, defining the closed stop plane of the door 132 relative to the door jamb 804.

A magnetic seal strip 808 is disposed on the face of the door stop strip 806 adjacent to the door edge 802. The strip 808 comprises a magnetic closure element configured to provide a positive magnetic attraction force drawing the door edge 802 into full compression contact with the seal assembly upon closure of the door 132.

The compression gasket 302 comprising a neoprene elastomeric gasket is disposed between the magnetic seal strip 808 and the door edge 802. The gasket 302 is mounted within a channel on the door stop strip 806 or the door edge 802 and is configured to compress upon closure of the door 132 to form a continuous acoustically sealed interface between the door edge 802 and the door jamb 804 assembly.

In the fully closed position, the neoprene compression gasket 302 is fully compressed between the door stop strip 806 and the door edge 802, and the magnetic seal strip 808 is in direct contact with the metal receptor to form a dual-element acoustic seal at the door perimeter.

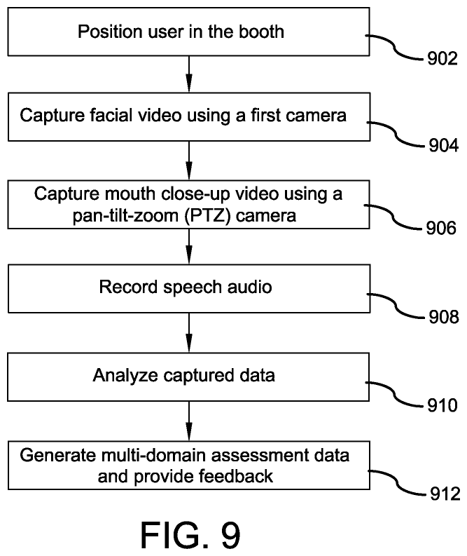

FIG. 9 illustrates a flowchart depicting a method of performing multi-domain speech assessment using the speech therapy booth in accordance with an embodiment of the present invention. At step 902, a user is positioned within the interior space of the speech therapy booth 100 in the seated position at the desk 118, with the door 132 closed and acoustically sealed. The speech therapy booth 100 establishes the acoustically isolated environment required for accurate speech capture and assessment.

At step 904, a first video stream of the user's face is captured using the webcam 114, providing full-face frontal video data for use in facial expression analysis, eye contact tracking, pragmatic behavior assessment, and video conferencing with a remote speech-language pathologist.

At step 906, a second video stream comprising a close-up view of the user's mouth and oral articulators is captured using the PTZ camera 116. The pan-tilt-zoom capability of the PTZ camera 116 is employed to obtain high-resolution imagery of the lips, teeth, tongue, and jaw of the user for articulation pattern analysis and oral motor function assessment.

At step 908, speech audio of the user is recorded within the acoustically isolated interior space of the speech therapy booth 100 using the USB microphone 134. The soundproof construction of the speech therapy booth 100 eliminates ambient noise interference, providing a clean audio signal for accurate acoustic analysis.

At step 910, the captured data comprising the first video stream, the second video stream, and the recorded speech audio are analyzed by the central computing controller 502 and associated AI processing systems, wherein the analysis encompasses simultaneous multi-domain assessment domains including articulation and phonology, fluency, oral motor function, voice quality, receptive language, expressive language, and pragmatics (also referred to as the AHFORVEP framework in the present disclosure).

Finally, at step 912, multi-domain assessment data is generated from the analysis of step 910 and real-time feedback is provided to the user via the monitor 112 and speakers 126 of the speech therapy booth 100. Assessment reports are also made available to a remote speech-language pathologist via the network interface of the controller 502, completing the speech assessment session cycle of the speech therapy booth 100.

It should be noted that the speech therapy assessment system using the speech therapy booth 100 of the present invention is organized around the AHFORVEP framework which is an eight-domain clinical assessment structure for the field of speech-language pathology. The eight domains of the AHFORVEP framework are articulation and phonology, hearing, fluency, oral motor, receptive language, voice, expressive language, and pragmatics.

The articulation and phonology domain relates to the accurate production of individual speech sounds and phonological patterns during speech. In the present invention, this domain is assessed via real-time phoneme recognition and scoring using the audio stream captured by the USB microphone 134 within the acoustically isolated interior space of the speech therapy booth 100, in conjunction with close-up oral articulator imagery simultaneously captured by the PTZ camera 116.

The hearing domain concerns the user's auditory perception and processing capabilities. In the present invention, this domain is assessed via automated speech-in-noise and digits-in-noise screening protocols executed by the central computing controller 502, wherein calibrated audio stimuli are presented to the user through the speakers 126 or headset 124 of the speech therapy booth 100 and the user's responses are captured and scored by the controller 502.

The fluency domain concerns the flow and continuity of speech, including the detection, classification, and severity scoring of stuttering events. In the present invention, fluency assessment is performed via acoustic feature analysis and disfluency detection algorithms applied to the audio stream captured by the USB microphone 134.

The oral motor domain concerns the physical movement, coordination, strength, and range of motion of the speech structures. In the present invention, this domain is assessed via real-time facial landmark tracking applied to the high-resolution close-up video stream of the PTZ camera 116, enabling quantitative measurement of lip position and movement, jaw range of motion and symmetry, tongue protrusion and lateralization visibility, and dental occlusion during speech and non-speech oral motor tasks.

The receptive language domain relates to the user's understanding and comprehension of spoken and written language. In the present invention, this domain is assessed via structured task presentation on the monitor 112 of the speech therapy booth 100, wherein the central computing controller 502 presents standardized receptive language tasks to the user and records and analyzes response latency, response accuracy.

The voice domain relates to the quality, pitch, loudness, and resonance characteristics of the user's voice. In the present invention, voice quality assessment is performed via acoustic feature extraction algorithms applied to the clean audio signal captured by the USB microphone 134 within the soundproof environment of the speech therapy booth 100.

The expressive language domain relates to the user's production of language including vocabulary diversity, grammatical accuracy, syntactic complexity, and mean length of utterance. In the present invention, expressive language assessment is performed via automatic speech transcription of the user's utterances by the controller 502, followed by natural language processing and large language model analysis of the transcribed output.

The Pragmatics domain concerns the social and contextual use of language, including eye contact maintenance, conversational turn-taking, facial expression appropriateness, topic maintenance, and social communication effectiveness. In the present invention, pragmatic behavior assessment is performed via gaze direction tracking and facial expression recognition applied to the full-face video stream of the webcam 114, along with large language model processing executing on the controller 502 or on cloud-based processing resources accessible via the network interface of the controller 502.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “soundproof speech therapy booth”, “soundproof speech therapy apparatus”, “speech therapy booth system”, and “speech therapy system” are interchangeable and refer to the soundproof speech therapy booth and system 100 of the present invention.

Notwithstanding the forgoing, the soundproof speech therapy booth and system 100 of the present invention can be of any suitable configuration as is known in the art without affecting the overall concept of the invention, provided that it accomplishes the above stated objectives. One of ordinary skill in the art will appreciate that the soundproof speech therapy booth and system 100 shown in the FIGS. are for illustrative purposes only, and that many other configurations of the soundproof speech therapy booth and system 100 are well within the scope of the present disclosure. Although the dimensions of the soundproof speech therapy booth and system 100 are important design parameters for user convenience, the soundproof speech therapy booth and system 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A telehealth speech-language therapy booth comprising:
a speech therapy booth comprising a plurality of wall panels;
a telehealth link;
a monitor;
a webcam; and
a pan-tilt-zoom (PTZ) camera;
wherein each wall panel of said plurality of wall panels comprises an acoustic foam panel;
wherein said each wall panel comprises a foam insulation layer for soundproofing;
wherein said monitor is mountable within an interior space of said speech therapy booth;
wherein said monitor comprises said telehealth link for session content selected from the group consisting of real-time AI-assisted speech assessment feedback, therapy exercises, and video conferencing content to a user within said speech therapy booth;
wherein said webcam configured to capture one or more selected from the group consisting of a full-face video stream of the user, a real-time facial expression analysis, eye contact tracking, and said video conferencing content during a speech therapy session; and
further wherein said pan-tilt-zoom (PTZ) camera is mounted on an interior wall of said speech therapy booth at a camera mounting height positioned to provide an angled view toward the user.

2. The telehealth speech-language therapy booth of claim 1, wherein said PTZ camera comprises a pan-tilt controller with a network connection and is configured to capture high-resolution close-up video imagery of the user's oral articulators selected from the group consisting of lips, teeth, tongue, and jaw.

3. The telehealth speech-language therapy booth of claim 2, wherein said imagery of the user's oral articulators provide one or more selected from the group consisting of articulation pattern analysis, oral motor function assessment, lip position tracking, jaw movement analysis, and tongue visibility detection during the speech therapy session.

4. The telehealth speech-language therapy booth of claim 3 further comprising a desk to provide a work surface for a keyboard and a mouse assembly disposed on a top surface of said desk for user interaction with a computing system of said speech therapy booth.

5. The telehealth speech-language therapy booth of claim 3 further comprising a speaker configured to deliver audio output from a remote speech-language pathologist to the user within said speech therapy booth.

6. The telehealth speech-language therapy booth of claim 5 further comprising a microphone as an audio capture device for recording the speech of the user within the soundproof interior space of said speech therapy booth.

7. The telehealth speech-language therapy booth of claim 6 further comprising a ventilation system configured to provide continuous airflow through the soundproof interior space of said speech therapy booth.

8. The telehealth speech-language therapy booth of claim 7 further comprising an exhaust fan configured to expel heated air from the interior space of said speech therapy booth to the exterior environment through a sound-baffled exhaust.

9. The telehealth speech-language therapy booth of claim 8 further comprising an air intake vent with a fan positioned diagonally opposite to said exhaust fan to promote diagonal cross-ventilation airflow traversing the interior space of said speech therapy booth.

10. The telehealth speech-language therapy booth of claim 1, wherein said webcam provides a frontal full-face capture axis directed perpendicularly from a wall panel toward the user and said PTZ camera provides a lateral angled capture axis directed from another wall panel toward the user's oral articulators, and further wherein said webcam and said PTZ camera capture axes together provide simultaneous full-face and oral articulator video capture during the speech therapy session.

11. The telehealth speech-language therapy booth of claim 10 further comprising a vibration isolation pad interposed between a base of said plurality of wall panels of said speech therapy booth and a foundation of said speech therapy booth.

12. A telehealth therapy booth comprising:
a therapy booth comprising a plurality of wall panels;
a telehealth link;
a monitor;
a webcam; and
a pan-tilt-zoom (PTZ) camera;
wherein said monitor is mountable within an interior space of said therapy booth;
wherein said monitor comprises said telehealth link for session content selected from the group consisting of real-time AI-assisted speech assessment feedback, therapy exercises, and video conferencing content to a user within said therapy booth;
wherein said webcam configured to capture one or more selected from the group consisting of a full-face video stream of the user, a real-time facial expression analysis, eye contact tracking, and said video conferencing content during a therapy session; and
further wherein said pan-tilt-zoom (PTZ) camera is mounted on an interior wall of said therapy booth at a camera mounting height positioned to provide an angled view toward the user.

13. The telehealth therapy booth of claim 12, wherein each wall panel of said plurality of wall panels comprises an acoustic foam panel, and further wherein said each wall panel comprises a foam insulation layer for soundproofing.

14. The telehealth therapy booth of claim 12, wherein said PTZ camera comprises a pan-tilt controller with a network connection and is configured to capture high-resolution close-up video imagery of the user's oral articulators selected from the group consisting of lips, teeth, tongue, and jaw.

15. The telehealth therapy booth of claim 14, wherein said imagery of the user's oral articulators provide one or more selected from the group consisting of articulation pattern analysis, oral motor function assessment, lip position tracking, jaw movement analysis, and tongue visibility detection during the therapy session.

16. The telehealth therapy booth of claim 12 further comprising a speaker configured to deliver audio output from a remote speech-language pathologist to the user within said therapy booth.

17. The telehealth therapy booth of claim 16 further comprising a microphone as an audio capture device for recording the speech of the user within the soundproof interior space of said therapy booth.

18. The telehealth therapy booth of claim 12, wherein said webcam provides a frontal full-face capture axis directed perpendicularly from a wall panel toward the user and said PTZ camera provides a lateral angled capture axis directed from another wall panel toward the user's oral articulators, and further wherein said webcam and said PTZ camera capture axes together provide simultaneous full-face and oral articulator video capture during the therapy session.

19. A method of performing telehealth therapy, the method comprising the steps of:
providing a therapy booth comprising a plurality of wall panels, a telehealth link, a monitor, a webcam, and a pan-tilt-zoom (PTZ) camera;
mounting said monitor within an interior space of said therapy booth;
wherein said monitor comprises said telehealth link for session content selected from the group consisting of real-time AI-assisted speech assessment feedback, therapy exercises, and video conferencing content to a user within said therapy booth;
capturing with said webcam one or more selected from the group consisting of a full-face video stream of the user, a real-time facial expression analysis, eye contact tracking, and said video conferencing content during a therapy session;
mounting said pan-tilt-zoom (PTZ) camera on an interior wall of said therapy booth at a camera mounting height positioned to provide an angled view toward the user; and
wherein said PTZ camera comprises a pan-tilt controller with a network connection and is configured to capture high-resolution close-up video imagery of the user's oral articulators selected from the group consisting of lips, teeth, tongue, and jaw.

20. The method of performing telehealth therapy of claim 19, wherein said webcam provides a frontal full-face capture axis directed perpendicularly from a wall panel toward the user and said PTZ camera provides a lateral angled capture axis directed from another wall panel toward the user's oral articulators, and further wherein said webcam and said PTZ camera capture axes together provide simultaneous full-face and oral articulator video capture during the therapy session.
